(19) United States
(12) Patent Application Publication
(10) Pub. No.: US 2023/0201149 A1
(43) Pub. Date: Jun. 29, 2023

(54) METHODS OF ANTIPATHOGENIC TREATMENT

(71) Applicant: CROFT INFRASTRUCTURE DESIGNS PTY LTD, Victoria (AU)

(72) Inventor: Terry Fortescue Croft, Victoria (AU)

(21) Appl. No.: 17/927,269

(22) PCT Filed: May 27, 2021
(86) PCT No.: PCT/AU2021/050515

§ 371 Date: Nov. 22, 2022

(30) Foreign Application Priority Data
May 27, 2020 (AU) 2020901738

(51) Int. Cl. A61K 31/198; A61K 31/385; A61K 38/53; A61K 33/04; A61K 36/064; A61K 33/30; A61K 31/375; A61K 31/593; A61K 31/51; A61K 33/06; A61P 31/14

(52) U.S. Cl. CPC A61K 31/198; A61K 31/385; A61K 38/53; A61K 33/04; A61K 36/064; A61K 33/30; A61K 31/375; A61K 31/593; A61K 31/51; A61K 33/06; A61P 31/14

(57) ABSTRACT

The present disclosure relates to methods for preventing, treating and/or reducing the severity of conditions or diseases associated with coronavirus infections. The methods comprise of administering an effective amount of a pharmaceutically acceptable compound which is an antioxidant or a substance capable of increasing the level of glutathione in the body. Preferably, the compound is a glutathione precursor selected from cysteine or a derivative thereof, cystine or a derivative thereof, methylsulfonylmethane (MSM) and dimethyl sulfoxide (DMSO), in particular, the glutathione precursor is N-acetylcysteine (NAC). In some embodiments, the compound is selected from lipoic acid, glycine, glutamate, or a derivative thereof, or a pharmaceutically acceptable salt or solvate thereof.

### CROSS-REFERENCE

The present application claims priority from Australian Provisional Patent Application No. 2020901738 filed on 27 May 2020, the entire contents of which are incorporated herein by reference.

### TECHNICAL FIELD

The disclosure relates to the field of antipathogenic therapy, in particular to new methods of antiviral treatment. The present disclosure is directed to methods and uses of pharmaceutically acceptable compounds, and compositions comprising the pharmaceutically acceptable compounds, for preventing, treating and/or reducing the severity of conditions or diseases associated with a viral infection.

### BACKGROUND

SARS-CoV-2 infection took over the human population in 2020 and remains a major health problem. The associated Coronavirus Disease 2019 (COVID-19), which was declared a pandemic by the World Health Organization (WHO) on 11 Mar. 2020, can have severe symptoms and is associated with the need for limited ICU health care resources. The disease is especially problematic for elderly patients, and goes on to be without effective treatment to date.

One of the common features of coronavirus infections is a significant morbidity and mortality associated with lung injury, pneumonia and acute respiratory distress syndrome (ARDS). In addition to these direct consequences, coronavirus infections also lead to a range of dangerous secondary effects. For example, COVID-19 can predispose patients to venous and arterial thromboembolic disease, hypoxia, immobilisation, diffuse intravascular coagulation, and thrombotic complications. Coronavirus infection is a risk factor for many further conditions, such as cytokine storm, venous or arterial thromboembolism, hypoxia, immobilisation, diffuse intravascular coagulation, symptomatic acute pulmonary embolism (PE), deep-vein thrombosis, ischemic stroke, myocardial infarction, systemic arterial embolism, reticular infiltration of the lungs, alveolar damage, coronary heart disease, asthma, obstructive pulmonary disease, sepsis and septic shock.

Health authorities around the world have been struggling to produce an effective treatment protocol. To date, recommendations of the authorities are mainly focused on respiratory support, while disease-modifying treatments or chemoprophylaxis have been largely based on drug repurposing and have seen minimal success. Potential chemotherapeutics include the anti-rheumatoid arthritis drug baricitinib, corticosteroids, a broad antiviral drug remdesivir, and tocilizumab; and further include azithromycin, convalescent plasma, hydroxychloroquine, hydroxychloroquine plus azithromycin, Interferon β-1a, and Interferon β-1a plus lopinavir-ritonavir, none of the which in the latter list are recommended.

Without a working therapeutic solution, most efforts have been concentrated on producing a vaccine to prevent the spread of coronavirus. Indeed, a vaccine to prevent infection with SARS-CoV-2 is currently regarded as the most effective way to halt the current COVID-19 pandemic. However, effective vaccine development, production and administration has been a challenging, costly and lengthy process, and the number of existing patients with the disease continues to be high and rising across the world.

Despite the advances in the understanding of the virus and its pathogenic attributes, and a plethora of efforts to combat COVID-19, current therapeutic strategies have persistently failed to achieve success, not being able to effectively reduce mortality rate, especially for elderly patients.

Therefore, there remains an urgent need for new or improved methods of preventing, alleviating or treating conditions, diseases or symptoms related to coronavirus infections.

It is to be understood that reference to any document in this application is not an admission that such document is available as prior art to the present invention. Any documents cited or referenced herein, as well as any product specifications, and product sheets for any products mentioned herein, are hereby incorporated herein by reference, and may be employed in the practice of the present invention.

### SUMMARY

The present inventor has discovered that administering effective doses of an antioxidant and/or any compound that results in increased levels of glutathione in the body reduces the symptoms of, or treats, a disease or condition that has been caused by or is associated with a coronavirus infection.

Accordingly, the present invention provides, in one aspect, a method of preventing, treating and/or reducing the severity of a condition or disease associated with a coronavirus infection in a subject, comprising administering to the subject an effective amount of a pharmaceutically acceptable compound; wherein the pharmaceutically acceptable compound is an antioxidant or a substance capable of increasing the level of glutathione in the subject.

In some embodiments, the coronavirus is a Betacoronavirus. In some embodiments, the coronavirus is selected from the group comprising Severe acute respiratory syndrome-related coronavirus 2 (SARS-CoV-2), Severe acute respiratory syndrome-related coronavirus (SARS-CoV), Middle East respiratory syndrome-related coronavirus (MERS-CoV), Human coronavirus OC43 (HCoV-OC43), Human coronavirus HKU1 (HCoV-HKU1), Human coronavirus 229E (HCoV-229E) and Human coronavirus NL63 (HCoV-NL63), and subtypes or variants thereof. In preferred embodiments, the coronavirus is severe acute respiratory syndrome coronavirus 2 (SARS-CoV-2) or a subtype or a variant thereof.

In some embodiments, the condition or disease is selected from coronavirus disease 2019 (COVID-19), severe acute respiratory syndrome (SARS), Middle East respiratory syndrome (MERS), pneumonia, acute respiratory distress syndrome (ARDS), cytokine storm, venous or arterial thromboembolism, hypoxia, immobilisation, diffuse intravascular coagulation, symptomatic acute pulmonary embolism (PE), deep-vein thrombosis, ischemic stroke, myocardial infarction, systemic arterial embolism, reticular infiltration of the lungs, alveolar damage, coronary heart disease, asthma, obstructive pulmonary disease, sepsis and septic shock.

In some embodiments, the pharmaceutically acceptable compound is formulated as a pharmaceutical composition comprising a pharmaceutically acceptable excipient.

In some embodiments, the pharmaceutically acceptable compound is administered orally, intravenously, subcutaneously, intramuscularly, intraperitoneally, sublingually, buccally, intratracheally, or by inhalation. In preferred embodiments, the pharmaceutically acceptable compound is administered intravenously.

In preferred embodiments, the pharmaceutically acceptable compound is one or more of cysteine or a derivative thereof, cystine or a derivative thereof, glutathione or a derivative thereof, a glutathione precursor, or an agent that can enhance the production of glutathione in vivo. Preferably, the pharmaceutically acceptable compound is a glutathione precursor.

In some embodiments, the glutathione precursor is a sulphur compound, preferably an organic sulphur compound that can be processed into glutathione in vivo.

In some embodiments, the glutathione precursor is selected from the group comprising cysteine or a derivative thereof, cystine or a derivative thereof, methylsulfonylmethane (MSM), and dimethyl sulfoxide (DMSO).

In preferred embodiments, the pharmaceutically acceptable compound is cysteine or a derivative thereof. Preferably, the pharmaceutically acceptable compound is the pharmaceutically acceptable compound is selected from L-cysteine, N-acetylcysteine (NAC), and glutamylcysteine, or a pharmaceutically acceptable salt or solvate thereof. Preferably, the pharmaceutically acceptable compound is N-acetylcysteine (NAC), or a pharmaceutically acceptable salt or solvate thereof. Most preferably, the N-acetylcysteine (NAC) is N-acetyl-L-cysteine, or a pharmaceutically acceptable salt or solvate thereof.

In some embodiments, the pharmaceutically acceptable compound is administered as a bolus intravenous injection or a continuous intravenous infusion. Preferably, the pharmaceutically acceptable compound is administered as a continuous intravenous infusion.

In some embodiments, N-acetylcysteine (NAC), or a pharmaceutically acceptable salt or solvate thereof, is administered at 1 mg or more per kg of body weight per 24 hours. Preferably, N-acetylcysteine (NAC), or a pharmaceutically acceptable salt or solvate thereof, is administered at 20 or more mg per kg of body weight per 24 hours; preferably 40 mg or more per kg of body weight per 24 hours; preferably 40 mg to 100 mg per kg of body weight per 24 hours; most preferably 40 mg per kg of body weight per 24 hours.

In some embodiments, N-acetylcysteine (NAC), or a pharmaceutically acceptable salt or solvate thereof, is administered at 60 mg per kg of body weight per 24 hours. In some embodiments, N-acetylcysteine (NAC), or a pharmaceutically acceptable salt or solvate thereof, is administered at 80 mg per kg of body weight per 24 hours. Preferably, N-acetylcysteine (NAC), or a pharmaceutically acceptable salt or solvate thereof, is administered at 100 mg per kg of body weight per 24 hours. In some examples, N-acetylcysteine (NAC), or a pharmaceutically acceptable salt or solvate thereof, is administered at 150 mg per kg of body weight per 24 hours.

In some embodiments, N-acetylcysteine (NAC), or a pharmaceutically acceptable salt or solvate thereof, is administered for 6 hours to 6 days or longer; preferably for 2 days to 8 days; preferably for 2 days to 6 days; preferably for 2 days to 4 days.

In some embodiments, the pharmaceutically acceptable compound is an agent that can enhance the production of glutathione in vivo.

In some embodiments, the agent is selected from one or more of lipoic acid, glycine, glutamate, or a derivative thereof, or a pharmaceutically acceptable salt or solvate thereof.

In some embodiments, the agent is a substance that upregulates an enzyme involved in production of glutathione in vivo. Preferably, the enzyme is selected from one or more of glutamate cysteine ligase, glutathione synthetase, and glutathione reductase.

In some embodiments, the pharmaceutically acceptable compound is administered in combination with an additional active agent.

In some embodiments, the additional active agent comprises a therapeutic agent suitable for use against a coronavirus infection. Preferably, the therapeutic agent is selected from Nafamostat, Remdesivir, Aprotinin, Nelfinavir, or a pharmaceutically acceptable salt or solvate thereof.

In some embodiments, the additional active agent comprises glycine or a derivative, pharmaceutically acceptable salt or solvate thereof. In some embodiments, the glycine or a derivative, pharmaceutically acceptable salt or solvate thereof is administered in an amount of 50 mg per day to 15 g per day; preferably 200 mg per day to 8 g per day. Preferably, the glycine or a derivative, pharmaceutically acceptable salt or solvate thereof is administered in an amount of at least 500 mg per day.

In some embodiments, the additional active agent comprises one or more of selenium, sodium selenite, selenium yeast, or a derivative, pharmaceutically acceptable salt or solvate thereof. In some embodiments, the one or more of selenium, sodium selenite, selenium yeast, or a derivative, pharmaceutically acceptable salt or solvate thereof is administered in an amount of elemental selenium of 50 μg per day to 400 μg per day; preferably 150 μg per day to 250 μg per day.

In some embodiments, the additional active agent comprises one or more of zinc, zinc gluconate, or a derivative, pharmaceutically acceptable salt or solvate thereof. In some embodiments, the one or more of zinc, zinc gluconate, or a derivative, pharmaceutically acceptable salt or solvate thereof is administered in an amount of elemental zinc of 20 mg per day to 300 mg per day; preferably 100 mg per day to 200 mg per day.

In some embodiments, the additional active agent further comprises one or more of vitamin C, vitamin D, magnesium, and thiamine (vitamin B1), or a pharmaceutically acceptable salt or solvate thereof.

In some embodiments, the subject is elderly, having pneumonia, or is otherwise vulnerable. Preferably, the subject is elderly.

In another aspect, the present invention provides a use of an effective amount of a pharmaceutically acceptable compound in the manufacture of a medicament for preventing, treating and/or reducing the severity of a condition or disease associated with a coronavirus infection, wherein the pharmaceutically acceptable compound is an antioxidant or a substance capable of increasing the level of glutathione in the subject.

In another aspect, the present invention provides an effective amount of a pharmaceutically acceptable compound for use in a method of preventing, treating and/or reducing the severity of a condition or disease associated with a coronavirus infection, wherein the pharmaceutically acceptable compound is an antioxidant or a substance capable of increasing the level of glutathione in the subject.

### BRIEF DESCRIPTION OF DRAWINGS

FIG. 1 is taken from Ibrahim et al. (2020) and depicts the effect of intravenous N-acetylcysteine on total and direct bilirubin levels in a COVID-19 patient. Shaded areas represent intervals of intravenous N-acetylcysteine administration. Initiation and termination of CC-ECMO are indicated along the horizontal axis.

FIG. 2 is taken from Ibrahim et al. (2020) and depicts the effect of intravenous N-acetylcysteine on C-reactive protein (CRP) and ferritin levels in a COVID-19 patient. Shaded areas represent intervals of intravenous N-acetylcysteine administration. Initiation and termination of CC-ECMO are indicated along the horizontal axis.

FIG. 3 is taken from Ibrahim et al. (2020) and depicts the effect of intravenous N-acetylcysteine on neutrophil/lymphocyte ratio (NLR) in a COVID-19 patient. Shaded areas represent intervals of intravenous N-acetylcysteine administration. Initiation and termination of CC-ECMO are indicated along the horizontal axis.

### DETAILED DESCRIPTION

### General Techniques and Definitions

Unless specifically defined otherwise, all technical and scientific terms used herein shall be taken to have the same meaning as commonly understood by one of ordinary skill in the art (e.g. medicine, antipathogenic therapy, pharmaceutically acceptable compounds, methods of antiviral treatment).

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features.

The present disclosure is not to be limited in scope by the specific embodiments described herein, which are intended for the purpose of exemplification only. Functionally equivalent products, compositions and methods are clearly within the scope of the disclosure, as described herein.

Each feature of any particular aspect or embodiment of the present disclosure may be applied mutatis mutandis to any other aspect or embodiment of the present disclosure.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

The term “subject” as used herein refers to a human or animal organism. Thus, the methods described herein are applicable to both human and veterinary disease. In certain embodiments, subjects are “patients,” i.e., living humans that are receiving medical care for a disease or condition. This includes persons with no defined illness who are being investigated for signs of pathology, as well as subjects who have an existing diagnosis of a particular viral disease which is being targeted by the compositions and methods of the present invention. Preferred viral diseases for treatment with the methods and uses described herein are diseases or conditions associated with an infection by a coronavirus. Methods described herein are suitable for subjects of any age, but are particularly advantageous for subjects who are elderly, having pneumonia, or are otherwise vulnerable, such as immunocompromised subjects. “Elderly” subjects refers to patients exhibiting an age of at least 50 years, more preferably of at least 55 years, 60 years, 65 years, 70 years, or older.

As used herein, the term “coronavirus” includes any member of the family Coronaviridae, including, but not limited to, any member of the genus coronavirus. The term “coronavirus” further includes naturally-occurring (e.g. wild-type) coronavirus; naturally-occurring coronavirus variants; and coronavirus variants generated in the laboratory, including variants generated by selection, variants generated by chemical modification, and genetically modified variants (e.g. coronavirus modified in a laboratory by recombinant DNA methods).

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease.

“An effective amount” is defined as a therapeutically effective amount of a reagent or pharmaceutical composition that is sufficient to show a patient benefit, i.e., to cause a decrease, prevention, or amelioration of the symptoms of the condition being treated. Effective amounts of the pharmaceutical formulation will vary according to factors such as the degree of susceptibility of the individual, the age, gender, and weight of the individual, and idiosyncratic responses of the individual. “Effective amount” encompasses, without limitation, an amount that can ameliorate, reverse, mitigate, prevent, or diagnose a symptom or sign of a medical condition or disorder or a causative process thereof. Unless dictated otherwise, explicitly or by context, an “effective amount” is not limited to a minimal amount sufficient to ameliorate a condition.

As used herein, “derivatives” are molecules that have been altered, for example by chemical conjugation or complexing with other chemical moieties (e.g., acylation and the like), modification (e.g., adding, removing or altering glycosylation), lipidation and/or inclusion of additional amino acid sequences as would be understood in the art. “Derivative”, as used herein, encompasses a prodrug. As used herein, the term “prodrug” refers to a molecule which can be converted to a pharmaceutically or therapeutically active compound upon chemical or enzymatic modifications of their structure. Generally, prodrug compounds are designed to be converted to drugs in vivo (e.g., in a target cell or target organ), such as upon administration to a subject. Accordingly, in some embodiments, the pharmaceutically acceptable compound is provided as a prodrug. A suitable prodrug includes a pharmaceutically acceptable derivatives of the compound which may be, for example, an ester, ether, amide, carbamate, phosphate, anhydride, or sulfonamide of the compound. Prodrugs can modify the physicochemical, biopharmaceutical, and pharmacokinetic properties of drugs. Traditional prodrugs are classified as drugs that are activated by undergoing transformation in vivo to form the active drug. Reasons for prodrug development are typically poor aqueous solubility, chemical instability, low oral bioavailability, lack of blood brain barrier penetration, and high first pass metabolism associated with the parent drug. Suitable prodrug moieties are described in, for example, “Prodrugs and Targeted Delivery,” J. Rautico, Ed., John Wiley & Sons, 2011.

### Methods of Treatment by Controlling Redox and Glutathione Levels

Without being bound by theory or mode of action, the present inventor has determined that coronaviruses may lead to a reduction in the levels of circulating Natural Killer (NK) cells, and the reduced levels of circulating NK cells may be directly responsible for the progression and severity of COVID-19. The present inventor has also determined that in SARS-CoV-2/COVID-19 patients, a T-helper type 2 (Th2) immune response predominates over a Th1 response. A high Th2 response is overall inferior at combatting pathogenic infections, and is associated with fatal outcomes.

Activation of NK cells is regulated by redox levels and glutathione; while a decrease in Th1 cytokine production correlates with a depletion of glutathione. Accordingly, the present inventor has surprisingly determined that detrimental effects of a coronavirus infection can be prevented or counteracted by administration of an antioxidant or a substance capable of increasing glutathione levels in the body.

The present inventor has found that by administering a compound that increases the level of glutathione in vivo, and/or administering an antioxidant, harmful pathological symptoms or consequences of a coronavirus infection can be prevented, alleviated or eliminated. In some cases, viral loads of coronavirus can be lowered. In some cases, patients suffering from a coronavirus infection can be completely cleared of the virus.

Accordingly, in one aspect, the present disclosure provides a method of preventing, treating and/or reducing the severity of a condition or disease associated with a coronavirus infection in a subject, comprising administering to the subject an effective amount of a pharmaceutically acceptable compound; wherein the pharmaceutically acceptable compound is an antioxidant or a substance capable of increasing the level of glutathione in the subject.

In the description provided herein, the Betacoronavirus SARS-CoV-2 or a variant thereof is exemplified. However, the disclosed methods are useful for the treatment, including prophylaxis, of any coronavirus infection, such as an infection by, for example, Severe acute respiratory syndrome-related coronavirus (SARS-CoV), Middle East respiratory syndrome-related coronavirus (MERS-CoV), Human coronavirus OC43 (HCoV-OC43), Human coronavirus HKU1 (HCoV-HKU1), Human coronavirus 229E (HCoV-229E) and Human coronavirus NL63 (HCoV-NL63), and subtypes or variants thereof.

The present disclosure provides a method of treating a condition or disease associated with a coronavirus infection, such as the coronavirus disease 2019 (COVID-19). However, coronavirus infections, including infections by SARS-CoV-2, are associated with a number of symptoms, conditions and diseases. In some embodiments, the condition or disease is the common cold, severe acute respiratory syndrome (SARS), Middle East respiratory syndrome (MERS), coronavirus disease 2019 (COVID-19), preferably coronavirus disease 2019 (COVID-19). In some embodiments, the condition or disease is pneumonia, for example COVID-19 pneumonia. In some embodiments, the condition or disease is acute respiratory distress syndrome (ARDS). The condition or disease may also be, for example, a cytokine storm, venous or arterial thromboembolism, hypoxia, immobilisation, diffuse intravascular coagulation, symptomatic acute pulmonary embolism (PE), deep-vein thrombosis, ischemic stroke, myocardial infarction, systemic arterial embolism, reticular infiltration of the lungs, alveolar damage, coronary heart disease, asthma, obstructive pulmonary disease, sepsis and septic shock.

The present disclosure provides a method of preventing, treating and/or reducing the severity of a condition or disease associated with a coronavirus infection. The methods generally involve administering an effective amount of a pharmaceutically acceptable compound. In some embodiments, the compound therapy is administered to a subject prophylactically, e.g., therapy is initiated before the appearance of symptoms. Such prophylactic treatment is administered in the case of individuals who are asymptomatic and who may or may not yet be infected, but who have come into close contact with an individual who has been diagnosed with e.g. COVID-19; individuals who are asymptomatic and who are not yet be infected, but who expect to come into contact with an individual who has been diagnosed with COVID-19 (e.g., health care workers working in a facility in which individuals who have been diagnosed with COVID-19 are being cared for); individuals who are asymptomatic and who are not yet be infected, and who are traveling to a location known to have a relatively high incidence of COVID-19 cases; and the like. Preferably, the compound therapy is administered to subjects who are elderly, having pneumonia, or are otherwise vulnerable such as being immunocompromised. Most preferably, the methods described herein are used in elderly human subjects, preferably subjects exhibiting an age of at least 50 years, more preferably of at least 55 years, 60 years, 65 years, 70 years, or older.

In other embodiments, the compound therapy is initiated after the appearance of clinical signs of a coronavirus infection, e.g. the clinical signs of COVID-19. Common signs and symptoms of COVID-19 include fever, cough, tiredness, loss of taste or smell, shortness of breath or difficulty breathing, chest pain, fever, sore throat, cough, difficulty breathing or shortness of breath, respiratory insufficiency, bronchitis, muscle pain, chest pain or pressure, dyspnea, pneumonia, acute respiratory distress syndrome (ARDS). An advantage of the disclosed methods is that the severity of symptoms is reduced, e.g., the viral load is reduced, and/or the time to viral clearance is reduced, and/or the morbidity or mortality is reduced, and/or the associated detrimental effects or conditions are prevented or alleviated.

### Antioxidant or a Substance Capable of Increasing the Level of Glutathione

In any of the above-described methods, a pharmaceutically acceptable compound is administered, which is an antioxidant or a substance capable of increasing the level of glutathione in vivo.

Any known antioxidant can be used in the present invention. The term “antioxidant” as used herein refers to a pharmaceutically acceptable compound which has anti-oxidative properties and which is suitable for alleviating oxidative stress and/or inflammation in a mammal. Antioxidant compounds include dietary supplements for protection against the effects of oxidative stress, e.g. ascorbic acid (vitamin C). Suitable antioxidants can react with free radicals directly and become self-oxidized; or act as reducing agents, e.g., cysteine. In some embodiments, antioxidants are enzymatic, e.g. superoxide dismutase (SOD), catalase (CAT), and glutathione peroxidase (GPX). Preferred antioxidants comprise cysteine and glutathione, or pharmaceutically acceptable derivatives, salts, complexes, or solvates thereof.

As used herein, the phrase “pharmaceutically acceptable salt” refers to pharmaceutically acceptable organic or inorganic salts. Exemplary acid addition salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. Exemplary base addition salts include, but are not limited to, ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethylpropylamine, or a mono-, di- or trihydroxy lower alkylamine, for example mono-, di- or triethanolamine. In some embodiments, the pharmaceutically acceptable salt is a sodium salt. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion. It will also be appreciated that non pharmaceutically acceptable salts also fall within the scope of the present disclosure since these may be useful as intermediates in the preparation of pharmaceutically acceptable salts or may be useful during storage or transport.

Those skilled in the art of organic and/or medicinal chemistry will appreciate that many organic compounds can form complexes with solvents in which they are reacted or from which they are precipitated or crystallized. These complexes are known as “solvates”. For example, a complex with water is known as a “hydrate”. As used herein, the phrase “pharmaceutically acceptable solvate” or “solvate” refer to an association of one or more solvent molecules and a compound of the present disclosure. Examples of solvents that form pharmaceutically acceptable solvates include, but are not limited to, water, isopropanol, ethanol, methanol, DMSO, ethyl acetate, acetic acid, and ethanolamine. It will be understood that the present disclosure encompasses solvated forms, including hydrates, of the compounds of the present disclosure and salts thereof.

The pharmaceutically acceptable compounds used in the methods of the present disclosure may exist as isomers, tautomers, racemates, stereoisomers, enantiomers and diastereoisomers. Asymmetric centres may exist in the compounds and compounds disclosed herein. These centres can be designated by the symbols “R” or “S,” depending on the configuration of substituents around the chiral carbon atom. It should be understood that the present disclosure encompasses all stereochemical isomeric forms, including diastereomeric, enantiomeric, and epimeric forms, as well as D-isomers and L-isomers, and mixtures thereof. Furthermore, the compounds disclosed herein may exist as tautomers or zwitterions, and all such isomers are within the scope of the present disclosure.

Any substance that can increase the level of glutathione in the body can be used in the present invention, including glutathione itself or a derivative thereof. For example, the substance may be a suitable glutathione precursor, such as cysteine, glutamate, or glycine, or pharmaceutically acceptable derivatives, salts, complexes or solvates thereof. Preferred examples of glutathione precursors include N-acetylcysteine (NAC), glutamylcysteine, and lipoic acid (a.k.a. α-lipoic acid, alpha-lipoic acid (ALA) and thioctic acid), or pharmaceutically acceptable derivatives, salts, complexes, or solvates thereof. Levels of glutathione may also be increased, for example, with a sulphur compound, preferably an organic sulphur compound that can be processed into glutathione in vivo, such as cysteine or a derivative thereof, cystine or a derivative thereof, methylsulfonylmethane (MSM), methionine, and dimethyl sulfoxide (DMSO), preferably cysteine or a derivative thereof.

In some embodiments, levels of glutathione may be increased by a substance which is an agent that can enhance the production of glutathione in vivo. Such agents can include, for example, feedstock building blocks for the formation of glutathione in the body, e.g. lipoic acid, glycine, glutamate, or a derivative thereof, or a pharmaceutically acceptable salt or solvate thereof, or another suitable organic sulphur compound such as MSM, dimethyl sulfoxide, and hydrogen sulphide. Further examples include reducing agents, such as NADPH. Alternatively, in some embodiments, the agent may be a compound that upregulates an enzyme involved in the production of glutathione in vivo. Enzymes involved in formation of glutathione in the body include, for example, glutamate cysteine ligase, glutathione synthetase, and glutathione reductase.

In some embodiments, the pharmaceutically acceptable compound is one or more of cysteine or a derivative thereof, cystine or a derivative thereof, glutathione or a derivative thereof, a glutathione precursor, or an agent that can enhance the production of glutathione in vivo. In a preferred embodiment, the pharmaceutically acceptable compound comprises cysteine or a pharmaceutically acceptable derivative, salt, complex, or solvate thereof; preferably N-acetylcysteine (NAC) or a pharmaceutically acceptable salt, complex, or solvate thereof; most preferably N-acetyl-L-cysteine or a pharmaceutically acceptable salt, complex, or solvate thereof.

N-acetylcysteine is an extremely safe drug that can be purchased over the counter at chemists or health food shops, for example under the tradename ACETADOTE®. Acetadote is supplied as a sterile solution in vials containing 20% w/v (200 mg/mL) N-acetyl-L-cysteine. The pH of the solution ranges from 6.0 to 7.5. Acetadote contains the following inactive ingredients: 0.5 mg/mL disodium edetate, sodium hydroxide (used for pH adjustment), and Sterile Water for Injection, USP.

N-acetylcysteine is currently used for treating acetaminophen overdose at very high doses. To treat acetaminophen overdose in adults based on the FDA approved three-bag method, N-acetylcysteine is administrated intravenously, initially 150 mg/kg in 200 mL of 5% dextrose for 60 minutes (loading dose), followed by 50 mg/kg in 500 mL of 5% dextrose for 4 hours (second dose), followed by 100 mg/kg in 1000 mL of 5% dextrose for 16 hours (third dose) (ACETADOTE® (acetylcysteine) Injection. NDA 21-539/S-004. FDA. 2006).

When administered orally, the bioavailability of N-acetylcysteine is only about 6-10%. Thus, in preferred embodiments, N-acetylcysteine is administered intravenously. Alternatively, in other embodiments, the bioavailability of orally administered N-acetylcysteine can be nearly doubled by utilizing liposomal N-acetylcysteine as described by the publication of Mitsopoulos and Suntres (2011), the content of which is incorporated herein by reference.

N-acetylcysteine administered intravenously is rapidly converted into glutathione. For example, Solton-Sharifi et al (2007) administered patients with 150 mg/kg of N-acetylcysteine diluted in 5% dextrose and infused over a period of 20 minutes on the first day and continued with 50 mg/kg/day diluted in 5% dextrose for three days.

The half-life of N-acetylcysteine in the body is relatively short, from about 2 h for reduced N-acetylcysteine to about 6 h for total N-acetylcysteine (Olsson et al. (1988)). Therefore, in preferred embodiments, N-acetylcysteine is administered as a continuous intravenous infusion. Alternatively, N-acetylcysteine may be administered orally at regular intervals.

### Pharmaceutical Compositions

In any of the above-described methods, one or more of the pharmaceutically acceptable compounds described above can be administered alone or as part of a pharmaceutical composition or formulation comprising one or more pharmaceutically acceptable diluents, carriers or excipients (collectively referred to herein as “excipient” materials).

The pharmaceutical compositions or formulations may for example be suitable for human medical use. The excipient(s) must be pharmaceutically acceptable in the sense of being compatible with the other ingredients of the formulation and not unduly deleterious to the recipient thereof.

Examples of pharmaceutical compositions or formulations include those suitable for oral, parenteral (including intravenous, intravitreal, subcutaneous, sublingual, buccal, intradermal, intratracheal, and intramuscular), inhalation, rectal, intraperitoneal, and topical administration.

For oral preparations, the one or more pharmaceutically acceptable compounds can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The one or more pharmaceutically acceptable compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

In some embodiments, compositions comprising the one or more pharmaceutically acceptable compounds are formulated for administration by parenteral delivery. For example, in one embodiment, the composition may be a sterile, lyophilized composition that is suitable for reconstitution in an aqueous vehicle prior to injection or infusion. For example, the composition may be a reconstituted composition produced by admixing of a solid composition as discussed above with a diluent such as saline or WFI (water for injection). Formulations for parenteral administration include aqueous and non-aqueous sterile injections solutions which may contain anti-oxidants, buffers, bacteriostats, and solutes that render the formulation isotonic with the blood of the intended recipient; and aqueous and non-aqueous sterile suspensions which may include suspending agents and thickening agents.

Exemplary compositions or parenteral administration include injectable solutions or suspensions which can contain, for example, suitable non-toxic, parenterally acceptable diluents or solvents, such as mannitol, 1,3-butanediol, water, dextrose, Ringer's solution, an isotonic sodium chloride solution, or other suitable dispersing or wetting and suspending agents, including synthetic mono or diglycerides, and fatty acids, including oleic acid.

In some embodiments, the composition comprises a liposome, a micelle, or a droplet. In some embodiments, the composition is an emulsion. Suitable emulsion constituents are known to the person skilled in the art, and generally comprise a mixture of an aqueous solution and a lipid solubiliser. In some embodiments, the emulsion formulation is formed with one or more lipid solubilisers selected from the group comprising a monoglyceride of a fatty acid (including 1-monoacylglycerols and 2-monoacylglycerols) or a diglyceride of a fatty acid, wherein the fatty acid moiety can be saturated or unsaturated. The lipid solubiliser can be a propane-1,2-diol ester of one or more fatty acids, such as propylene glycol heptanoate, propylene glycol monocaprylate, propylene glycol dilaurate, propylene glycol monocaprylate, propylene glycol monolaurate, or others. In some embodiments, glutathione or a precursor thereof is formulated as liposomal formulation.

### Administration and Dosage

In the methods described herein, one or more of the pharmaceutically acceptable compounds described above may be administered to a subject using any convenient means capable of resulting in the desired therapeutic effect. Thus, the compounds can be incorporated into a variety of formulations for therapeutic administration. More particularly, the agents of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants and aerosols.

As such, administration of the one or more pharmaceutically acceptable compound can be achieved in various ways, including oral, buccal, sublingual, rectal, parenteral, intraperitoneal, intradermal, subcutaneous, intramuscular, transdermal, intranasal, pulmonary, intratracheal, etc., administration. In some embodiments, the one or more compound is administered orally, preferably orally at regular intervals. In some embodiments, two different routes of administration are used. In some embodiments, the one or more compound is administered parenterally. In preferred embodiments, the one or more compound is administered intravenously or by inhalation, preferably intravenously. In preferred embodiments, the one or more compound is administered as a bolus intravenous injection or a continuous intravenous injection, most preferably as a continuous intravenous injection.

Intravenous administration of the one or more pharmaceutically acceptable compound is accomplished using standard methods and devices. In some embodiments, the one or more compound is administered by a continuous delivery system. Mechanical or electromechanical infusion pumps can also be suitable for use with the present invention. Particularly preferred is administration by continuous intravenous infusion.

The one or more compounds can be administered daily, twice daily, every other day, twice a week, three times a week, or substantially continuously or continuously. In some embodiments, the one or more compounds is administered orally at regular intervals. In some embodiments, administration is by continuous intravenous infusion. Effective dosages of the one or more pharmaceutically acceptable compound can range from about 1 mg to about 300 mg per kg of body weight per 24 hours, preferably from about 10 mg to about 250 mg per kg of body weight per 24 hours; preferably from about 20 mg to about 200 mg per kg of body weight per 24 hours; preferably from about 30 mg to about 180 mg per kg of body weight per 24 hours; preferably from about 40 mg to about 160 mg per kg of body weight per 24 hours; preferably from about 50 mg to about 150 mg per kg of body weight per 24 hours; preferably from about 60 mg to about 140 mg per kg of body weight per 24 hours; preferably from about 70 mg to about 130 mg per kg of body weight per 24 hours; preferably from about 80 mg to about 120 mg per kg of body weight per 24 hours; preferably from about 90 mg to about 120 mg per kg of body weight per 24 hours; preferably from about 100 mg to about 110 mg per kg of body weight per 24 hours.

In some embodiments, the pharmaceutically acceptable compound is N-acetylcysteine, most preferably N-acetyl-L-cysteine, or a pharmaceutically acceptable salt, complex, or solvate thereof. Effective dosages of N-acetylcysteine range from at least about 1 mg per kg of body weight per 24 hours; at least about 5 mg per kg of body weight per 24 hours; at least about 10 mg per kg of body weight per 24 hours; at least about 20 mg per kg of body weight per 24 hours; at least about 30 mg per kg of body weight per 24 hours; preferably at least about 40 mg per kg of body weight per 24 hours; preferably at least about 50 mg per kg of body weight per 24 hours; preferably at least about 60 mg per kg of body weight per 24 hours; preferably at least about 70 mg per kg of body weight per 24 hours; preferably at least about 80 mg per kg of body weight per 24 hours; preferably at least about 90 mg per kg of body weight per 24 hours; preferably at least about 100 mg per kg of body weight per 24 hours; preferably at least about 110 mg per kg of body weight per 24 hours; preferably at least about 120 mg per kg of body weight per 24 hours; preferably at least about 130 mg per kg of body weight per 24 hours; preferably at least about 140 mg per kg of body weight per 24 hours; preferably at least about 150 mg per kg of body weight per 24 hours. N-acetylcysteine can be administered several times a day, daily, every other day, three times a week, or substantially continuously or continuously; preferably substantially continuously or continuously.

In preferred embodiments, N-acetylcysteine is administered to a subject intravenously in a dosage of from about 10 mg to about 150 mg per kg of body weight; from about 10 mg to about 150 mg per kg of body weight; from about 10 mg to about 150 mg per kg of body weight; from about 10 mg to about 150 mg per kg of body weight; from about 10 mg to about 150 mg per kg of body weight; from about 10 mg to about 150 mg per kg of body weight; from about 10 mg to about 150 mg per kg of body weight; from about 10 mg to about 150 mg per kg of body weight. In particularly preferred embodiments, the dose is about 20 mg per kg of body weight per 24 hour; 30 mg per kg of body weight per 24 hour; 40 mg per kg of body weight per 24 hour; 50 mg per kg of body weight per 24 hour; 60 mg per kg of body weight per 24 hour; 70 mg per kg of body weight per 24 hour; 80 mg per kg of body weight per 24 hour; 90 mg per kg of body weight per 24 hour; 100 mg per kg of body weight per 24 hour; 110 mg per kg of body weight per 24 hour; 120 mg per kg of body weight per 24 hour; 130 mg per kg of body weight per 24 hour; 140 mg per kg of body weight per 24 hour; 150 mg per kg of body weight per 24 hour.

In some embodiments, for example for prophylactic or preventative purposes, N-acetylcysteine may be administered orally at a dose of about 600 mg. Prophylactic dosages of oral N-acetylcysteine include about 1000 mg; about 900 mg; about 800 mg; about 700 mg; about 600 mg; about 500 mg, about 400 mg; about 300 mg of drug per dose. In some embodiments, for example for patients having symptoms, at least about 1200 mg of oral N-acetylcysteine may be administered to alleviate symptoms and accelerate recovery from virus infection; preferably at least about 1300 mg, at least about 1400 mg; at least about 1500 mg; at least about 1600 mg; at least about 1700 mg; at least about 1800 mg; at least about 1900 mg; at least about 2000 mg of drug per dose.

In some embodiments, N-acetylcysteine may be administered for 6 hours to 8 days or longer; for 12 hours to 8 days or longer; for 24 hours to 8 days or longer; for 2 days to 8 days or longer; for 3 days to 8 days or longer. In some embodiments, N-acetylcysteine may be administered for 2 days to 8 days; preferably for 2 days to 7 days; preferably for 2 days to 6 days; preferably for 2 days to 5 days; most preferably for 2 days to 4 days.

In some embodiments, the one or more pharmaceutically acceptable compound is administered in a first dosing regimen (also referred to as “the induction regimen”), followed by a second dosing regimen. The first dosing regimen of one or more pharmaceutically acceptable compound generally involves administration of a higher dosage of the one or more pharmaceutically acceptable compound. For example, in the case of intravenous N-acetylcysteine, the first dosing regimen comprises administering N-acetylcysteine at about 150 mg per kg of body weight; 140 mg per kg of body weight; 130 mg per kg of body weight; 120 mg per kg of body weight; 110 mg per kg of body weight; 100 mg per kg of body weight. In some embodiments, the first doing regimen achieves N-acetylcysteine concentration in the blood of about 1 mM. The first dosing regimen can encompass a single dosing event, or at least two or more dosing events. The first dosing regimen of the one or more pharmaceutically acceptable compound can be administered daily, every other day, three times a week, every other week, three times per month, once monthly, substantially continuously or continuously. For example, in the case of N-acetylcysteine, the first dosing regimen can be administered by continuous intravenous infusion.

The second dosing regimen of the one or more pharmaceutically acceptable compound (also referred to as “the maintenance dose”) generally involves administration of a lower amount of the one or more pharmaceutically acceptable compound. For example, in the case of intravenous N-acetylcysteine, the second dosing regimen comprises administering N-acetylcysteine at a dose of at least about at least about 10 mg per kg of body weight; at least about 20 mg per kg of body weight; at least about 30 mg per kg of body weight; preferably at least about 40 mg per kg of body weight; preferably at least about 50 mg per kg of body weight; preferably at least about 60 mg per kg of body weight; preferably at least about 70 mg per kg of body weight; preferably at least about 80 mg per kg of body weight; preferably at least about 90 mg per kg of body weight; preferably at least about 100 mg per kg of body weight. The second dosing regimen can encompass a single dosing event, or at least two or more dosing events. The second dosing regimen of the one or more pharmaceutically acceptable compound can be administered daily, every other day, three times a week, every other week, substantially continuously or continuously.

For example, N-acetylcysteine can be infused at a dose of 100 mg kg of body weight for at least 3 days. In other embodiments, for example in case of severe symptoms of a coronavirus infections such as, for example, when a subject develops ARDS, N-acetylcysteine can be administered at 150 mg/kg on the first day, followed by a dose of 100 mg per kg of body weight per day, for at least 1 day, at least 2 days, preferably at least 3 days, at least 4 days, at least 5 days, preferably at least 6 days, at least 7 days, or at least 8 days.

Those of skill in the art will readily appreciate that dose levels can vary as a function of the specific compound, the severity of the symptoms and the susceptibility of the subject to side effects. Preferred dosages for a given compound are readily determinable by those of skill in the art by a variety of means.

### Additional Therapeutic Agents

The above-described one or more pharmaceutically acceptable compounds can be administered in combination with an additional active agent.

In some embodiments, any of the above-described treatments are used in conjunction with administration of one or more therapeutic agent that is suitable or convenient for treating a pathological coronavirus infection, or is hypothesised or suspected to be a potential therapy for a coronavirus infection. Additional antiviral agents that are suitable for use in combination therapy include, but are not limited to, nucleotide and nucleoside analogs. Non-limiting examples include AZT (zidovudine), DDI (didanosine), DDC (dideoxycytidine), D4T (stavudine), combivir, abacavir, adefovir dipoxil, cidofovir, ribavirin, hydroxychloroquine, Nafamostat, Remdesivir, Nelfinavir, Aprotinin, or a pharmaceutically acceptable salt or solvate thereof, and the like. In some embodiments, the antiviral agent is hydroxychloroquine. Preferred antiviral agents include, but are not limited to, Nafamostat, Remdesivir, and Nelfinavir, or a pharmaceutically acceptable derivative, salt, complex, or solvate thereof.

In some embodiments, the methods further include administration of Nafamostat. The invention also contemplates use of pharmaceutically acceptable derivatives or salts of Nafamostat, such as Nafamostat Mesylate. Nafamostat may be administered orally in capsule or tablet form, or in the same or different administration form and in the same or different route as the one or more pharmaceutically acceptable compound. Of course, other types of administration of both medicaments, as they become available are contemplated, such as by nasal spray, transdermally, intravenously, by suppository, by sustained release dosage form, etc. Any form of administration will work so long as the proper dosages are delivered without destroying the active ingredient.

In some embodiments, Nafamostat is administered in an amount ranging from about 0.01 to about 5 mg/kg body weight per hour, or about 0.05 to about 2.5 mg/kg body weight per hour, or about 0.1 to about 1 mg/kg body weight per hour, or about 0.1 to about 0.5 mg/kg body weight per hour, preferably about 0.1 to about 0.2 mg/kg body weight per hour, or about 1 to about 10 mg/kg body weight per day, or about 2 to about 8 mg/kg body weight per day, preferably about 2 to about 5 mg/kg body weight per day.

In some embodiments, the methods further include administration of Remdesivir, or a derivative, pharmaceutically acceptable salt, complex or solvate thereof. In some embodiments, Remdesivir is administered by continuous intravenous infusion. In some embodiments, Remdesivir may be administered for example at about 200 mg for the first 24 h, then for example at about 100 mg per 24 h or up to 10 mg/kg body weight per day, either as a single infusion or at a constant infusion. For example, Remdesivir may be administered individually or in conjunction with Omeprazole, for example with Omeprazole at a plasma concentration of up to about 1 to about 10 μM, preferably about 8 μM. Remdesivir may be administered in the same or different administration form and in the same or different route as the one or more pharmaceutically acceptable compound.

In some embodiments, the methods further include administration one or more of Cepharanthine, Niclosamide, and Aprotinin, or a derivative, pharmaceutically acceptable salt, complex or solvate thereof, which may be administered, for example, by drip infusion. In some embodiments, one or more of these agents are administered alone or in conjunction with Ciclesonide and/or Nitazoxanide.

In some embodiments, an additional antiviral agent is administered during the entire course of treatment with the one or more pharmaceutically acceptable compounds described above. In other embodiments, an additional antiviral agent is administered for a period of time that is overlapping with that of the treatment with the one or more compound, e.g., the additional antiviral agent treatment can begin before the compound treatment begins and end before the compound treatment ends; the additional antiviral agent treatment can begin after the compound treatment begins and end after the compound treatment ends; the additional antiviral agent treatment can begin after the compound treatment begins and end before the compound treatment ends; or the additional antiviral agent treatment can begin before the compound treatment begins and end after the compound treatment ends.

In some embodiments, any of the above-described treatments are used in conjunction with administration of one or more therapeutic agent that is selected from the group including but not limited to glycine, selenium, sodium selenite, selenium yeast, zinc, zinc gluconate, zinc ionophore, zinc-saturated lactoferrin, lactoferrin, iron, vitamin C, vitamin D, magnesium, thiamine (vitamin B1), whey protein, quercetin, Epigallocatechin-gallate (EGCG), nasal and oral drugs, sodium bicarbonate, or a derivative, pharmaceutically acceptable salt, complex or solvate thereof. In preferred embodiments, the one or more agent is selected from glycine, selenium, sodium selenite, selenium yeast, zinc, zinc gluconate, zinc ionophore, zinc-saturated lactoferrin, lactoferrin, vitamin C, vitamin D, magnesium, and thiamine (vitamin B1), or a derivative, pharmaceutically acceptable salt, complex or solvate thereof.

In some embodiments, the methods further include administration of glycine, or pharmaceutically acceptable derivatives, salts, complexes or solvates thereof. In some embodiments, the glycine is administered in an amount ranging from about 5 mg to about 50 g per day; preferably from about 10 mg to about 40 g per day; preferably from about 20 mg to about 30 g per day; preferably from about 30 mg to about 20 g per day; preferably from about 50 mg to about 15 g per day; preferably from about 100 mg to about 10 g per day; preferably from about 200 mg to about 8 g per day. In some embodiments, the glycine is administered in an amount of at least 50 mg per day; at least 100 mg per day; at least 200 mg per day; at least 300 mg per day; at least 400 mg per day; at least 500 mg per day.

In some embodiments, the methods further include administration of one or more of selenium, sodium selenite, selenium yeast, or pharmaceutically acceptable derivatives, salts, complexes or solvates thereof. In some embodiments, the one or more of selenium, sodium selenite, or selenium yeast is administered in an amount of elemental selenium ranging from about 5 μg to about 1000 μg per day; preferably from about 10 μg to about 900 μg per day; preferably from about 20 μg to about 700 μg per day per day; preferably from about 30 μg to about 600 μg per day per day; preferably from about 40 μg to about 500 μg per day per day; preferably from about 50 μg to about 400 μg per day per day; preferably from about 60 μg to about 350 μg per day; preferably from about 80 μg to about 325 μg per day; preferably from about 100 μg to about 300 μg per day; preferably from about 150 μg to about 250 μg per day.

In some embodiments, the methods further include administration of one or more of zinc, zinc gluconate, or pharmaceutically acceptable derivatives, salts, complexes or solvates thereof. In some embodiments, the one or more of zinc, zinc gluconate is administered in an amount of elemental zinc ranging from about 4 mg to about 1000 mg per day; preferably from about 6 mg to about 850 mg per day; preferably from about 8 mg to about 600 mg per day; preferably from about 10 mg to about 500 mg per day; preferably from about 15 mg to about 400 mg per day; preferably from about 20 mg to about 300 mg per day; preferably from about 40 mg to about 250 g per day; preferably from about 60 mg to about 230 mg per day; preferably from about 80 mg to about 220 mg per day; preferably from about 100 mg to about 200 mg per day.

In some embodiments, the methods further include administration of one or more of vitamin C, vitamin D, magnesium, and thiamine (vitamin B1), or pharmaceutically acceptable derivatives, salts, complexes or solvates thereof. These additional active agents can be administered orally in capsule or tablet form, or in the same or different administration form and in the same or different route as the one or more pharmaceutically acceptable compound. Other types of administration of the agents, as they become available are contemplated, such as by nasal spray, transdermally, intravenously, by suppository, by sustained release dosage form, etc. Any form of administration will work so long as the proper dosages are delivered without destroying the active ingredients.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

### EXAMPLES

### Example 1: Severe COVID-19 Patients Showed Improvement Upon Treatment with N-Acetylcysteine

A clinical study using N-acetylcysteine (NAC) to treat COVID-19 patients was performed by Ibrahim et al. (2020), published in the report entitled “Therapeutic blockade of inflammation in severe COVID-19 infection with intravenous N-acetylcysteine” on 22 Jul. 2020, and incorporated herein by reference.

Ibrahim and the co-workers demonstrated that intravenous NAC significantly improved COVID-19 disease conditions in a total of ten respirator-dependent COVID-19 patients aged 38 to 71, including one with a Glucose-6-phosphate dehydrogenase (G6PD) deficiency. Patients 1 and 2 received 30 g of intravenous NAC per day, while patients 3 to 10 were administered 600 mg intravenous NAC twice daily. Intravenous NAC administration significantly reduced inflammatory markers such as C-reactive protein (CRP) and ferritin, and also improved lung functions. Eight patients were eventually discharged, and two remaining patients showed improved conditions.

In the first part of the study, a 44-year-old man presented with a 5-day history of fever, cough, and shortness of breath. The patient was earlier diagnosed with G6PD deficiency after hemolytic reaction to sulfa drugs. The patient tested positive for SARS-CoV-2 by PCR. Upon admission, his inflammatory markers, such as C-reactive protein (CRP), ferritin, and D-dimer, neutrophil to lymphocyte ratio (NLR) were elevated. The patient's liver function tests, hemoglobin (Hb), and white blood cell count were normal. Patient was started on hydroxychloroquine on the next day and received only one dose of 400 mg. His respiratory status continued to worsen and required intubation on the 4^{th} day of admission. Despite intubation and maximum ventilation settings the patient respiratory status continued to deteriorate requiring veno-venous extracorporeal membrane oxygenator (VV ECMO) which was started on the 6^{th} day.

TABLE 1
Laboratory test values of G6PD-deficient patient upon
admission for COVID-19 infection before administration of
hydroxychloroquine (Ibrahim et al. (2020)).

| Variable | Admission | value Reference value |
|---|---|---|
| G6PD U/g Hemoglobin | 0.5 | >9 |
| White blood cells × 10³/μL | 5.3 | 4.2-9.1 |
| Hemoglobin mg/dL | 12.6 | 13.7-17.5 |
| Platelets × 10³/μL | 205 | 150-400 |
| Neutrophil % | 73 | 34-68 |
| Lymphocyte % | 18 | 22-53 |
| C-reactive protein mg/L | 45 | 0-5 |
| Ferritin ng/ml | 491 | 22-248 |
| D-dimer ng/ml | 520 | <230 |
| Bilirubin, total mg/dL | 1.0 | 0.2-1.2 |
| Bilirubin, direct mg/dL | 0.5 | 0-0.5 |
| Interlukin-6 pg/ml | 20 | <5 |

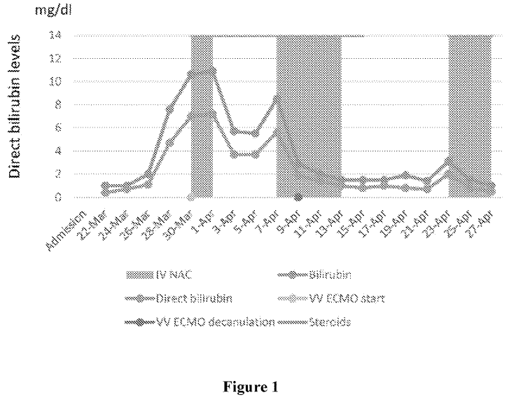

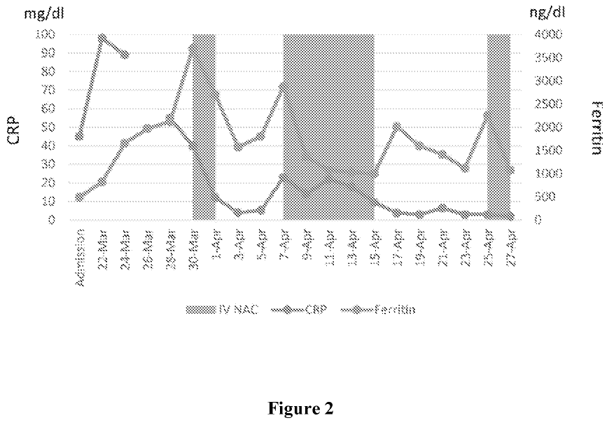

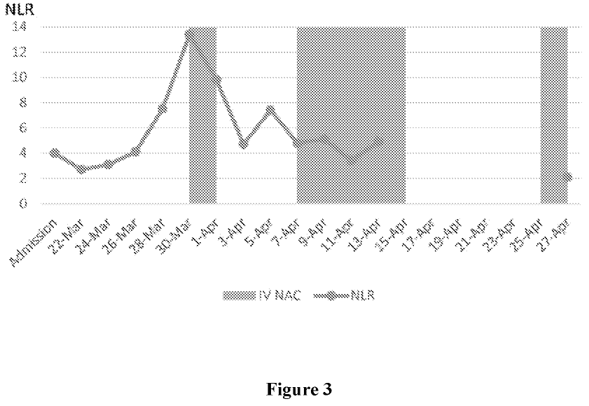

On the 9^{th} day, the patient's Hb level dropped (7.9 g/dL) and his direct (6.0 mg/dL) and total bilirubin had become dramatically elevated (9.0 mg/dL). Alanine aminotransferase (ALT) and aspartate aminotransferase (AST) peaked at 263 U/L and 338 U/L, respectively. Further testing revealed low G6PD level (0.5 U/g Hb) and haptoglobin (2 mg/dL). Blood smear showed bell cells suggesting G6PD-deficiency hemolysis. The patient was started on intravenous (IV) N-acetylcysteine on the 10^{th} day (30,000 mg divided into three doses over 24 h for just two day). This was followed by immediate improvement in hemolysis indices (FIG. 1). ALT and AST improved to 100 U/L and 62 U/L, respectively. One week after IV suggesting G6PD-deficiency hemolysis. The patient was started on intravenous (IV) N-acetylcysteine discontinuation, total and direct bilirubin started rising again and IV N-acetylcysteine was re-started at 600 mg every 12 h for six days. Again, IV N-acetylcysteine administration was associated with resolution of hemolysis as evident by sustained reduction in bilirubin (total and direct) (FIG. 1) and an increase in haptoglobin. Patient oxygenation continued to improve and his VV ECMO was discontinued. Ten days after discontinuation of the second round of IV N-acetylcysteine, a slight increase in both total and direct bilirubin was noted, IV N-acetylcysteine was started again on April 25th at 600 mg every 12 h, this again was associated with reduction in total and direct bilirubin (FIG. 1). Patient continued to improve clinically and was discharged to rehab on April 27th and was then discharged home on April 30th. Of note, the patient was treated with steroids starting March 30th. Notably, a reduction in inflammatory markers (CRP and ferritin) was observed that coincided with IV N-acetylcysteine administration (FIG. 2). Additionally, IV N-acetylcysteine was associated with a decrease in NLR that was sustained after the first dose (FIG. 3).

Due to this successful outcome, IV N-acetylcysteine was given to 9 further COVID-19 patients without G6PD deficiency. Eight of the nine patients required VV ECMO. A significant overall reduction in inflammatory markers (CRP and ferritin) was observed during IV N-acetylcysteine administration. A rebound of inflammation was noted in six patients following discontinuation of N-acetylcysteine. In the other three patients IV N-acetylcysteine was associated with decrease in CRP and ferritin without rebound increase after discontinuation. The median CRP level during IV N-acetylcysteine administration 55 mg/dL was significantly lower than during the periods without IV N-acetylcysteine (either before administration 143 mg/dL (46-235), or after IV NAC discontinuation 69 mg/dL (27-114).

The results of this study provide direct evidence of the effectivity of intravenous NAC in the treatment of severe COVID-19 patients.

### Example 2: COVID-19 Patients with Dyspnea were Effectively Treated with Glutathione, NAC and Lipoic Acid

Nasi A. et al. described, in a report entitled “Reactive oxygen species as an initiator of toxic innate immune responses in retort to SARS-CoV-2 in an ageing population, consider N-acetylcysteine as early therapeutic intervention” published on 18 Jun. 2020, that two COVID-19 patients with dyspnea were effectively treated with oral and IV glutathione, NAC and α-lipoic acid (Nasi A. (2020)).

### Example 3: NAC in Combination with a Low Dose Hydroxychloroquine had a Positive Impact on an Elderly COVID-19 Patient

Puyo et al. reported, in a manuscript entitled “Case Report: use of hydroxychloroquine and N-acetylcysteine for treatment of a COVID-19 positive patient” and published on 2 Jun. 2020, that using low dose hydroxychloroquine and intravenous NAC showed improvement on a 54 year old male COVID-19 patient who presented with multi-system organ damage. The patient was given a low dose oral hydroxychloroquine (total 600 mg) in combination with intravenous NAC, at a loading dose of 75 mg/kg for 4 hours, then 35 mg/kg for 16 hours, followed by 17 mg/kg for 24 hours. The patient gradually recovered (NLR from 16.7 to 2.4) despite pulmonary embolism and short term mechanical ventilation. The patient was then released from intensive care on day 7 and eventually discharged on post-admission day 12.

### Example 4: High Doses of Vitamin C, Zinc and Lactoferrin were Therapeutic in COVID-19 Patients

A study by Serrano et al. (2020) has shown, for example, that when liposomal bovine lactoferrin (dose between 128 and 192 mg/day) was administered with zinc (20-30 mg/day) and vitamin C (doses of 48-72 mg/day), all 12 patients recovered in the first 4-5 days of the study. In addition to effectivity of zinc and vitamin C treatment, this result shows that the high doses of these active agents could be safely tolerated for a significantly superior result.

### Example 5: Glycine and NAC Increased the Levels of Reduced Glutathione In Vivo

A clinical study by McCarty et al. (2018) found that red blood cell concentrations of glycine averaged 487 μmol/L in young subjects and 218 μmol/L in elderly subjects; but this level rose to 529 μmol/L in the elderly subjects after they were administered 100 mg/kg of both glycine and N-acetylcysteine daily for 14 days. Their red blood cell total glutathione levels (GSH+GSSG) rose from 1.26 mmol/L at baseline to 2.23 mmol/L, a concentration slightly higher than that measured in young subjects who did not take supplements. Moreover, the ratio of GSH to GSSG rose from 7.4:1 to 16.1:1, indicative of a substantial improvement in redox status.

### Example 6: Glutathione Levels were Raised by Administration of Glycine and NAC in Elderly Patients

A clinical study by Sekhar et al. (2011) investigated red blood cell concentrations in young control subjects (unsupplemented) and elderly subjects (before and after supplementation) with 100 mg/kg of both glycine and N-acetylcysteine for 14 days, producing the following results.

TABLE 2
(Sekhar et al. (2011))

| | Young control | Elderly subjects (n = 8) | |
|---|---|---|---|
| | subjects | Before | After |
| Variable | (n = 8) | supplementation | supplementation |
| Glycine (μmol/L) | 486.7 ± 28.3² | 218.0 ± 23.7³ | 528.6 ± 33.5 |
| Cysteine (μmol/L) | 26.2 ± 1.4⁴ | 19.8 ± 1.3⁵ | 30.6 ± 2.2 |
| Glutamate (μmol/L) | 463.1 ± 69.0 | 464.0 ± 115.3 | — |
| GSSG (mmol/L) | 0.11 ± 0.04⁴ | 0.15 ± 0.05 | 0.13 ± 0.05 |
| Glutathione:GSSG ratio | 18.9 ± 2.1² | 7.4 ± 2.3⁶ | 16.1 ± 4.3 |

The elderly subjects had a 44.9% slower glutathione fractional synthesis rate (FSR) (83.14±6.43 compared with 45.80±5.69%/d; P<0.01) and a 68.2% slower absolute synthesis rate (ASR) (1.73±0.16 compared with 0.55±0.12 mmol glutathione/L RBC/d; P<0.01) at baseline.

After treatment with cysteine and glycine for 14 d, compared with pre-supplementation values, the elderly subjects in the post-supplemented state had a 94.6% higher RBC glutathione concentration (from 1.12±0.18 to 2.18±0.35 mmol glutathione/L RBC; P<0.05) and a 78.8% higher FSR (from 45.80±5.69 to 81.91±7.70%/d; P<0.01), resulting in a 230.9% higher ASR (from 0.55±0.12 to 1.82±0.39 mmol glutathione/L RBC per day; P<0.01).

### Example 7: Selenium had Beneficial Effects in the Treatment of ARDS

A clinical study by Mahmoodpoor et al. (2019) found that intravenous selenium administered to ARDS patients resulted in a drop in IL-1beta (R value: −0.624; P<0.001) and IL-6 (R value: 0.642; P<0.001).

### Example 8: Vitamin D Subtype was Able to Reduce Severity of COVID-19

In a parallel pilot randomized open label, double-masked clinical trial study by Castillo et al. (2020), administration of a high dose of calcifediol or 25-hydroxyvitamin D, a main metabolite of vitamin D endocrine system, significantly reduced the need for ICU treatment of patients that required hospitalization due to COVID-19. The authors showed that calcifediol seemed to be capable of reducing the severity of the disease.

In the trial, eligible patients were allocated to take oral calcifediol 0.532 mg (roughly equivalent in potency to a dose of 68,000 IU of vitamin D), or not. Patients in the calcifediol treatment group continued with oral calcifediol (0.266 mg) on day 3 and 7, and then weekly until discharge or ICU admission. Outcomes of effectiveness included rate of ICU admission and deaths.

Of 50 patients treated with calcifediol, one required admission to the ICU (2%), while of 26 untreated patients, 13 required admission (50%). Of the patients treated with calcifediol, none died, and all were discharged without complications. The 13 patients not treated with calcifediol, who were not admitted to the ICU, were discharged. Of the 13 patients admitted to the ICU, two died and the remaining 11 were discharged.

### Example 9: Administration of Zinc had a Therapeutic Effect in COVID-19 Patients

A clinical study by Derwand et al. (2020) demonstrated that administration of zinc had a therapeutic effect in COVID-19 outpatients.

In an outpatient setting treated patients with zinc, (50 mg elemental zinc as zinc sulphate), low-dose hydroxychloroquine (200 mg twice daily) and azithromycin (500 mg once daily) (entitled triple therapy) dependent on risk stratification. The treatment was limited to five days.

The patients were stratified into three groups namely Group A, age >60 years, with or without clinical symptoms; Group B, age ≤60 years and shortness of breath (SOB); or Group C, age ≤60 years, clinically symptomatic and with at least one of the following co-morbidities: hypertension, hyperlipidaemia, diabetes mellitus, obesity [body mass index (BMI)≥30 kg/m2], cardiovascular disease, heart failure, history of stroke, history of deep vein thrombosis or pulmonary embolism, asthma, chronic obstructive pulmonary disease (COPD), other lung disease, kidney disease, liver disease, autoimmune disease or history of cancer. Pregnant women, if any, were also included in this group. Patients were not treated with HCQ if they had known contraindications, including QT prolongation, retinopathy or glucose-6-phosphate dehydrogenase deficiency.

Using this stratification 62% of COVID-19 patients were treated with standard of care only and recovered at home and only 38% needed treatment with the triple therapy.

The diagnosis of COVID-19 for all patients in this analysis was confirmed by PCR or IgG tests. Starting triple therapy as early as possible after symptom onset is critical for treatment success because SARS-CoV-2 viral load appears to peak at days 5-6 after symptom onset and severe cases progress to ARDS after only 8-9 days.

A median of 4 days after the onset of symptoms, 141 patients (median age 58 years, IQR 40-67 years; 73.0% male) received a prescription for triple therapy for 5 days. Independent public reference data from 377 confirmed COVID-19 patients in the same community were used as untreated controls. Of 141 treated patients, 4 (2.8%) were hospitalized, which was significantly fewer (P<0.001) compared with 58 (15.4%) of 377 untreated patients [odds ratio (OR)=0.16, 95% confidence interval (CI) 0.06-0.5]. One patient (0.7%) in the treatment group died versus 13 patients (3.4%) in the untreated group (OR=0.2, 95% CI 0.03-1.5; P=0.12). This one patient had a history of cancer and only took one daily dose of the triple therapy before hospital admission. No cardiac side effects were observed and no patient reported palpitations or any cardiac side effects.

### Example 10: A Combination of Vitamin D, Magnesium and Vitamin B Improved the Condition of Elderly COVID-19 Patients

A clinical study by Tan et al. (2020) demonstrated that a combination of vitamin D, magnesium and vitamin B improved the condition of elderly COVID-19 patients.

Patients were administered oral vitamin D3 1000 IU OD, magnesium 150 mg OD and vitamin B12 500 mcg OD (DMB) upon admission if they did not require oxygen therapy. Primary outcome was deterioration post-DMB administration leading to any form of oxygen therapy and/or intensive care support.

Results: 43 consecutive COVID-19 patients aged ≥50 were identified. 17 patients received DMB and 26 patients did not. Baseline demographic characteristics between the two groups was significantly different in age. In the treatment arm, most patients received DMB within the first day of hospitalization with a median duration of therapy of 5 days (interquartile range of 4 to 7 days). In univariate analysis, age and hypertension showed significant influence on outcome while DMB retained protective significance after adjusting for age or hypertension separately in multivariate analysis. Fewer DMB patients than controls required initiation of oxygen therapy during their hospitalization (17.6% vs 61.5%, P=0.006). DMB exposure was associated with odds ratios of 0.13 (95% CI: 0.03-0.59) and 0.20 (95% CI: 0.04-0.93) for oxygen therapy and/or intensive care support on univariate and multivariate analyses respectively.

Without being bound by theory, the present disclosure may also be described with reference to one or more of the following paragraphs.

Except for a small lipid phase the human body is a water based system governed by the law of thermodynamics. The most important parameter being the body's antioxidant couple, in particular the glutathione couple, GSH x GSH/GSSG. This treatment revolves around this theory.

There are numerous examples of significantly reduced glutathione level. One example is the significant reduced level in the elderly. It is speculated this is the reason for the increased morbidly and mortality of the COVID virus in this cohort. This deficiency can be overcome with appropriate supplements.

Glutathione, that regulates intracellular redox, regulates glutathione levels in antigen-presenting cells (APC) that in turn determine whether T helper cytokines, Th1 or Th2 response patterns predominate. More probably the actual control is the glutathione couple.

Increasing the glutathione content causes a preponderance of Th1 cells to form characterized by interleukin 12 (IL-12) and gamma interferon production and up-regulation of cell-mediated responses. The Th2 response patterns is characterized by IL-4 and IL-10 and the up-regulation of a variety of antibody responses when there is a deficiency of glutathione. The glutathione level can be raised by administering N-acetylcysteine, a precursor of glutathione.

In extreme cases immune response may develop exclusively in either a Th1 or Th2 response pattern. Considering the large and continuous infusion of NAC proposed one can expect all or nearly all the T helper cells will be Th1 ones.

Tests on aging mice showed they had a shift to Th2 response. This probably, at least partially, accounts for the higher mortality rate in the elderly. This effect will be eliminated with our proposed treatment.

With a Th1 response IL-12 is secreted physiologically by monocytes, Mp and dendritic cells in response to bacteria and bacterial products.

The Th1/Th2 balance is regulated by the balance between reductive macrophages (RMp) those with a high intracellular content of glutathione and oxidative macrophages (OMp) with a reduced content of glutathione. Also CD4+ CD44-naïve Th0 cells are differentiated preferentially either to Th1 or Th2, depending on the presence of RMp or OMp during the initial 24 h of culture.

Besides the interleukin suite noted above, RMp augment NO (nitric oxide) generation with decreased production of IL-6 while OMp augmented 11-6 production. Thus the alteration in Mp function because of the altered intracellular glutathione may play a relevant role in the pathological progression of inflammation.

Increased IL-6 is a known pathway to inflammation. Th2 polarization, in vitro, exacerbated airway inflammation in a murine model of allergic asthma. While an increase in GSH levels ameliorate bronchial asthma by altering the Th1/Th2 imbalance through IL-12 production. (i.e. a shift to Th1 cells)

Mps with elevated intracellular glutathione making them RMp elevated the capacity to produce IL-12 and nitric oxide (NO) and reduced release of IL-6, IL-10 and prostaglandin E2 (PGE2)

Considering the foregoing the large continuous dose of 8 gm/day of NAC should result in nearly all the T cells being Th1 releasing copious amounts of gamma IFN a potent killer of viruses to kill viruses, with reduced IL-6 there should reduced inflammation and the gamma IFN plus the nitric oxide should be a potent killer against any bacteria and other pathogens

IFN gamma has been clinically used to treat a wide variety of diseases. The original function of IFN gamma is its natural antiviral activity, and this molecule may be effective in viral infection and consequent disseminated multi-organ invasion. Despite its role as an inflammatory cytokine IFN gamma induces regulatory T cells and antigen-specific regulatory B cells, which play a counter-regulatory role in the immune reaction, possibly preventing or controlling excessive immune response such as cytokine storms that can result in death.

The advantages of IFN gamma are as follows: IFN gamma is a non-virus specific anti-viral therapeutic and can be used in new virus infections and epidemics; IFN gamma is strongly predicted to be effective in viral infection”.

These comments are for an exogenously applied therapeutic while the IFN gamma released due to the Th1 effect is likely to be released onto or in the virus and surely must be far more potent than an exogenous application.

The severity and outcome of the COVID-19 cases has been associated with the percentage of circulating lymphocytes (LYM %), levels of Interleukin6 (IL-6), C reactive protein (CRP) procalcitonin (PCT), lactic acid (LA) and viral load (ORFlab Ct).

LYM %, CRP and IL-6 are the most sensitive and reliable factors in distinguishing between survivors and non-survivors. LYM % is the most sensitive and reliable in discriminating between critically ill, severe and moderate types, and between survivors and non-survivors.

There are a number of pathways to inflammation arguably the most significant is the up-regulation of IL-6, a Th2/OMg cytokine.

The most significant source of IL-6 is monocytes and macrophages. This indicates there is a strong skewing of the T helper cells to Th2 and OMg macrophages in COVID infection.

There is no doubt significant reactive oxygen species being generated to combat the virus and related pneumonia and other associated infections that would be consuming glutathione and lowering the glutathione couple value and in turn skewing to Th2/OMg. Also the reduced level of glutathione in the elder patients as outline in the section on elderly patient would also be accentuating this skewing to Th2/OMg.

In keeping with this hypothesis, increased secretion of Th2 cytokines IL-4 and IL-10 have been observed.

With the N-acetylcysteine infusion there should be mainly Th1 T cells and RMg macrophages and very few IL-6 cytokines and in turn reduced inflammation.

If need be the inflammation can be blocked by the administering of tocilizumab.

Acute respiratory distress syndrome (ARDS) is the leading cause of mortality. They also noted secondary haemophagocytic lymphohistiocytosis (sHLH) is an under-recognised hyperinflammatory syndrome where IL-6 is even more significant as its p value is p<0.0001.

Fortuitously the high infusion of NAC with the proposed treatment will skew the T helper profile sharply to Th1 with little Th2 resulting in significantly less IL-6 being produced.

COVID-19 may predispose to both venous and arterial thromboembolism due to excessive inflammation, hypoxia, immobilisation and diffuse intravascular coagulation.

The incidence of the composite outcome of symptomatic acute pulmonary embolism (PE), deep-vein thrombosis, ischemic stroke, myocardial infarction or systemic arterial embolism has been determined.

All patients in the ICU received standard doses of thromboprophylaxis. 27% of these patients had venous thromboembolism (VTE) and another 3.7% had an arterial thrombotic event. Pulmonary embolism (PE) was the most frequent thrombotic complication. While this 31% is extremely high it is not known how small a percentage of the infected population were admitted to the ICUs

A comparison of the hematological parameters between the mild and severe groups showed significant differences in interleukin-6, (IL-6), d-Dimer, glucose, thrombin time, fibrinogen and C-reactive protein. It was determined IL-6 and d-Dimer levels could predict the severity of COVID up to 93%. This rapid test may aid early detection of the above complications.

Another study determined the cause of death of 12 patients. Coronary heart disease and asthma or chronic obstructive pulmonary disease were the most common comorbid conditions (50% and 25% respectively. Autopsy revealed deep venous thrombosis in 7 of the 12 patients in whom venous thromboembolism was not suspected before death; pulmonary embolism was the direct cause of death in 4 patients. Postmortem computed tomography revealed reticular infiltration of the lungs with severe bilateral dense consolidation, whereas histomorphologically diffuse alveolar damage was seen in 8 patients. In all patients, COVID-19 RNA was detected in the lungs at high concentrations; viremia in 6 of 10 and 5 of 12 patients demonstrated high viral RNA titers in the liver, kidney, or heart.

Heparin has been used to combat these complications and reduced the death rate from 64% to 40% in the most severe cases. There is no benefit for the less severe cases. Specifically the treatment appears to provide a better prognosis in severe COVID patients with coagulopathy.

It should also be noted that the expected low level of IL-6 due to skewing to Th1/RMg and the expected rapid killing of the virus, the anti-thrombotic properties of the anti-viral drug, Nafamostat with the addition of heparin if need be, these complications should not eventuate.

This severity of COVID-19 rises exponentially over the age of 60. This is due, at least partially, to the shift to Th2/OMp and the increase in IL-6. The predominance of Th2 cells is probably due to the marked decrease in glutathione levels with ageing.

The glutathione levels have been found to be just over 50% of younger subjects. Even more significant was the glycine level, one of the three amino acids that form glutathione and found it to be only 210 micromol/L compared to 486 micromol/L in the young subjects. Cysteine level was closer to young subjects at 19.8 micromol/L compared to 26.2 micromol/L. Cysteine is generally regarded as the limiting amino acid but there will be ample supplementation from the NAC infusion.

Zinc intake decreases the concentration of plasma high-sensitivity C-reactive protein (hsCRP), interleukin (IL)-6. macrophage chemoattractant protein 1 (MCP-1), vascular cell adhesion molecule 1 (VCAM-1), secretory phospholipase A2, and malondialdyde and hydroxyalkenals (MDA+HAE) in elderly subjects. While these finding were after six months of 45 mg zinc/day as a gluconate we speculate the effect should be substantially obtained within days. This supplementation should be part of the treatment.

Selenium supplementation can have a profound influence on systemic inflammatory response syndrome (SIRS) and multiple organ dysfunction syndrome (MODS) in its own capacity and in up regulation glutathione peroxidase (GPx). Patients with SIRS & MODS had a significant decrease in GPx-3 and Se. (P=0.0001 and P=0.002 respectively). Another example Alzheimer patients when given 200 microg/day reduced serum high sensitivity C-reactive protein (P<0.001) and significantly increased total glutathione (P=0.001). On these results elderly patients should be given up to 200 microg/day of selenium.

Type 2 diabetes (T2D) is a major comorbidity of COVID-19. However, the impact of blood glucose (BG) control on the degree of required medical interventions and the mortality in patients with COVID-19 and T2D remains uncertain.

Subjects with T2D require more medical interventions and have a significantly higher mortality (7.8% verses 2.7%; adjusted hazard ratio [HR]1.49) and multiple organ injury than the non-diabetic individuals. Furthermore well-controlled BG (glycemic variability within 3.9 to 10.0 mmol/L) was associated with markedly lower mortality compared to individuals with poorly controlled BG (upper limit of glycemic variability exceeding 10.0 mmol/L) (adjusted HR, 0.14) during hospitalization. These findings provide clinical evidence correlating improved improved glycemic control with better outcomes in patients with COVID-19 and pre-existing T2D.

A word of caution. This treatment especially the large N-acetlycysteine infusion is likely to reduce the diabetic effect and the quantity of any prescribed drugs will probably need to be reduced to prevent the patient becoming hypoglycemic.

Analysis of cancer in COVID-19 cases in China up to Jan. 31, 2020 reveled 18 (1.1% of 1590 cases) had a history of cancer, which seems to be higher than the national average of 0.29%.

Lung cancer was the most frequent type affecting 28% of the 18 patients. 25% had received chemotherapy or surgery within the past month and the other 75% were cancer survivors in routine follow up after primary resection. Patients with cancer were older (mean age 63.1 years v 48.7 years, more likely to have a history of smoking 22% compared with 7% for non smokers and had more polypnea, 47% compared to 23% of the other patients and more severe baseline CT manifestation, 94% compared 71% of other patients.

More importantly, patients with cancer were observed to have a higher risk of severe events (a composite endpoint defined as the percentage of patients being admitted to the intensive care unit requiring invasive ventilation or death) compared with patients without cancer, 39% vs 8% of other patients, Fisher's exact p=0.0003

Suitable Anti-Viral Drugs

Based on laboratory trials, Nafamostat is by far the most promising drug.

The genomic RNA of coronaviruses such as COVID-19 is surrounded by an envelope composed of a lipid bilayer and envelope proteins. COVID-19 initiates human cell entry after the Spike protein (S protein) present on the envelope binds to a cell membrane receptor, ACE2.(*2) The S protein is cleaved into S1 and S2 by a human cell-derived protease (proteolytic enzyme) that is assumed to be Furin. S1 then binds to its receptor, ACE2. The other fragment, S2, is cleaved by TMPRSS2 (*3) a human cell surface serine protease, resulting in membrane fusion.

(*2) ACE2: Angiotensin converting enzyme 2, which catalyzes the conversion of angiotensin II to angiotensin 1-7.

(*3) TMPRSS2: Transmembrane protease, serine 2. A serine protease present in the cell surface membrane. The COVID-19 coronavirus S protein is said to undergo proteolysis by TMPRSS2 after binding to the host receptor. Absent protein degradation, membrane fusion cannot proceed. Nafamostat is thought to inhibit S protein-initiated membrane fusion by inhibiting TMPRSS2 activity.

Earlier research of Nafamostat indicated it would not have sufficient concentration in the plasma to cause death of the virus. This was based on trialing drugs in monkey kidney cells. It was determined it would be more realistic to trial the drugs in human lung cells. The results were dramatic in that vastly different effects resulted. In particular Nafamostat 50% kill rate IC50 decreased 6000 fold to just 0.0022 microM, an exceptionally low figure indicating it was 600 fold more potent than Remdesivir.

Moreover although Nafamostat inhibits COVID-induced cytopathogenic effect (CPE) that is structural changes in host cells that are caused by viral invasion, it displayed limited effects on the virus replication cycle as indicated by high levels of double-strand RNA in Nafamostat-treated COVID-infected cells. This lack of killing of the virus by Nafomostat is not of concern as the IFN gamma induced by the skewing to Th1/RMg should be able to do this killing.

Nafamostat has an added benefit in that it is an anticoagulant adding the removal of blood clots.

Expected doses are 01-02 mg/kg/hr (2.4-4.8 mg/kg/day).

Nafamostat has been approved for human use in Japan and Korea for over a decade so it can be repurposed for COVID treatment.

Remdesivir has been shown to have some beneficial effect. The efficacy can be increased 10 fold by the addition of therapeutic concentrations of the proton pump inhibitor, omeprazole.

Aprotinin a serine protease inhibitor that is used in Russia for treating influenza and is applied as an aerosol may add further benefits as the omeprazole increases the effectiveness of aprotinin 2.7 fold.

Nelfinavir, an HIV-1 protese inhibitor, potently inhibits replication of SARS-Cov-2. The effective concentration for 50% and 90% inhibition (EC50 and EC90) of nelfinavir were 1.13 microM and 1.76 microM respectively, the lowest of the nine HIV-1 protese inhibitors trialed including lopinavir. The trough and peak serum concentration of nelfinavir were three to six times higher than the EC50 of this drug (lopinavir).

The present disclosure may also be defined with reference to one or more of the following numbered paragraphs.

1. To kill the pathogen an antioxidant is utilized.

2. The antioxidant is a substance that has a reduced state and an oxidized state that is administered in its reduced state in sufficient amount to shift the T helper (Th) cytokine to a Th1 state as opposed to a Th2 state and provide reductive macrophages (RMp) as opposed to oxidative macrophages (OMp).

3. The quantity of the antioxidant may be reduced depending on whether one wants to solely kill the pathogen with Th1 and RMp or whether one also wants antibodies to form utilizing Th2 and OMp.

4. The pathogens whether they are viruses, bacteria, fungi, protozoa or other pathogens are killed by the interferon gamma released by the Th1 cytokines and reduced macrophages and other actions performed by these cytokines and macrophages.

5. The antioxidant is a sulphur compound, preferably an organic sulphur compound.

6. The preferred compound is a derivative of cysteine/cystine such as N-acetlycysteine (NAC) or L-cysteine, Methylsulfonylmethane (MSM) or dimethyl sulfoxide (DMSO).

7. The preferred method of administering the antioxidant is intravenously at a constant flow rate although it may be administered orally at regular intervals.

8. The preferred antioxidant is N-acetylcysteine (NAC) administered at one milligram or more per kilogram of body weight per 24 hours. The preferred rate is 100 milligrams per kilogram of body weight per 24 hours.

9. This treatment can also be used to combat sepsis and septic shock.

10. To aid the killing of the pathogen another drug that specifically kills the pathogen will be utilized.

11. To aid the eradication of the COVID-19 virus one or more substances that diminish the virulence of the virus will be utilized.

12. Nafamostat Mesylate is one such substance. The preferred method of utilization is drip-infused intravenously at a constant rate of at least 1.0 microgram of nafamostat mesylate per kilogram of body weight per hour with the current preferred rate of about 0.15 milligram per kilogram of body weight per hour.

13. Alternatively or in addition Remdesivir at a preferred continuous intravenous infusion of 200 milligrams for the first 24 hours then 100 milligrams per 24 hours or up to 10 milligrams per kilogram of body weight per day either as a single infusion or at a constant infusion either individually or given orally and in conjunction with omeprazole at a plasma concentration up to about 8 micro molar.

14. Alternative or in addition nelfinavir along with cepharanthine or niclosamide administered by drip infusion alone of in conjunction with ciclesonide or nitazoxanide or aprotinin will be administered.

15. Where patients' glutathione levels are low especially in the elderly or due to pneumonia or other causes the glutathione levels will be raised by the NAC infusion plus a glycine supplement.

16. Elderly patients or those with pneumonia or other vulnerable patients 40 milligrams of zinc per day as zinc gluconate will be administered

17. Elderly patients or those with pneumonia or other vulnerable patients up to 200 micrograms of selenium per day will be administered.

### REFERENCES

Castillo M. E. et al. (2020) Journal of Steroid Biochemistry and Molecular Biology 203: 105751.
Derwand R. et al. (2020) International Journal of Antimicrobial Agentts 56(6) 106214.
Ibrahim, H. et al. (2020) Clinical Immunology 219:108544.
Mahmoodpoor A. et al. (2018) Immunological Investigations 48: 147-159.
McCarty M. F. et al. (2018) Ochsner Journal 18: 81-87.
Mitsopoulos, P. and Suntres, Z. E. (2011) Journal of Toxicology 2011: 808967.
Nasi A. et al. (2020) Toxicology Reports 7: 768-771.
Olsson B. et al. (1988) European Journal of Clinical Pharmacology 34: 77-82.
Puyo C. et al. (2020) F1000Research 9: 491.
Serrano G. et al. (2020) International Journal Of Research In Health Sciences 8(1): 8-15.
Sekhar R. V. et al. (2011) Am J Clin Nutr. 94(3): 847-853.
Tan C. W. et al. (2020) Nutrition 79: 111017.

1. A method of preventing, treating and/or reducing the severity of a condition or disease associated with a coronavirus infection in a subject, comprising administering to the subject an effective amount of a pharmaceutically acceptable compound;
wherein the pharmaceutically acceptable compound is an antioxidant or a substance capable of increasing the level of glutathione in the subject.

2. The method according to claim 1, wherein the coronavirus is a Betacoronavirus.

3. The method according to claim 1 or claim 2, wherein the coronavirus is selected from the group comprising Severe acute respiratory syndrome-related coronavirus 2 (SARS-CoV-2), Severe acute respiratory syndrome-related coronavirus (SARS-CoV), Middle East respiratory syndrome-related coronavirus (MERS-CoV), Human coronavirus OC43 (HCoV-OC43), Human coronavirus HKU1 (HCoV-HKU1), Human coronavirus 229E (HCoV-229E) and Human coronavirus NL63 (HCoV-NL63), and subtypes or variants thereof.

4. The method according to any one of claims 1 to 3, wherein the coronavirus is severe acute respiratory syndrome coronavirus 2 (SARS-CoV-2) or a subtype or a variant thereof.

5. The method according to any one of claims 1 to 4, wherein the condition or disease is selected from coronavirus disease 2019 (COVID-19), severe acute respiratory syndrome (SARS), Middle East respiratory syndrome (MERS), pneumonia, acute respiratory distress syndrome (ARDS), cytokine storm, venous or arterial thromboembolism, hypoxia, immobilisation, diffuse intravascular coagulation, symptomatic acute pulmonary embolism (PE), deep-vein thrombosis, ischemic stroke, myocardial infarction, systemic arterial embolism, reticular infiltration of the lungs, alveolar damage, coronary heart disease, asthma, obstructive pulmonary disease, sepsis and septic shock.

6. The method according to any one of claims 1 to 5, wherein the pharmaceutically acceptable compound is formulated as a pharmaceutical composition comprising a pharmaceutically acceptable excipient.

7. The method according to any one of claims 1 to 6, wherein the pharmaceutically acceptable compound is administered orally, intravenously, subcutaneously, intramuscularly, intraperitoneally, sublingually, buccally, intratracheally, or by inhalation.

8. The method according to any one of claims 1 to 7, wherein the pharmaceutically acceptable compound is administered intravenously.

9. The method according to any one of claims 1 to 8, wherein the pharmaceutically acceptable compound is one or more of cysteine or a derivative thereof, cystine or a derivative thereof, glutathione or a derivative thereof, a glutathione precursor, or an agent that can enhance the production of glutathione in vivo.

10. The method according to any one of claims 1 to 9, wherein the pharmaceutically acceptable compound is a glutathione precursor.

11. The method according to claim 10, wherein the glutathione precursor is a sulphur compound, preferably an organic sulphur compound that can be processed into glutathione in vivo.

12. The method according to claim 10 or claim 11, wherein the glutathione precursor is selected from the group comprising cysteine or a derivative thereof, cystine or a derivative thereof, methylsulfonylmethane (MSM), and dimethyl sulfoxide (DMSO).

13. The method according to any one of claims 1 to 12, wherein the pharmaceutically acceptable compound is cysteine or a derivative thereof.

14. The method according to any one of claims 1 to 13, wherein the pharmaceutically acceptable compound is selected from L-cysteine, N-acetylcysteine (NAC), and glutamylcysteine, or a pharmaceutically acceptable salt or solvate thereof.

15. The method according to any one of claims 1 to 14, wherein the pharmaceutically acceptable compound is N-acetylcysteine (NAC), or a pharmaceutically acceptable salt or solvate thereof.

16. The method according to claim 15, wherein the N-acetylcysteine (NAC) is N-acetyl-L-cysteine, or a pharmaceutically acceptable salt or solvate thereof.

17. The method according to any one of claims 1 to 16, wherein the pharmaceutically acceptable compound is administered as a bolus intravenous injection or a continuous intravenous infusion.

18. The method according to any one of claims 1 to 17, wherein the pharmaceutically acceptable compound is administered as a continuous intravenous infusion.

19. The method according to any one of claims 15 to 18, wherein N-acetylcysteine (NAC), or a pharmaceutically acceptable salt or solvate thereof, is administered at 1 mg or more per kg of body weight per 24 hours.

20. The method according to any one of claims 15 to 19, wherein N-acetylcysteine (NAC), or a pharmaceutically acceptable salt or solvate thereof, is administered at 20 or more mg per kg of body weight per 24 hours.

21. The method according to any one of claims 15 to 20, wherein N-acetylcysteine (NAC), or a pharmaceutically acceptable salt or solvate thereof, is administered at 40 mg or more per kg of body weight per 24 hours.

22. The method according to any one of claims 15 to 21, wherein N-acetylcysteine (NAC), or a pharmaceutically acceptable salt or solvate thereof, is administered at 40 mg to 100 mg per kg of body weight per 24 hours.

23. The method according to any one of claims 15 to 22, wherein N-acetylcysteine (NAC), or a pharmaceutically acceptable salt or solvate thereof, is administered at 60 mg per kg of body weight per 24 hours.

24. The method according to any one of claims 15 to 23, wherein N-acetylcysteine (NAC), or a pharmaceutically acceptable salt or solvate thereof, is administered at 80 mg per kg of body weight per 24 hours.

25. The method according to any one of claims 15 to 24, wherein N-acetylcysteine (NAC), or a pharmaceutically acceptable salt or solvate thereof, is administered at 100 mg per kg of body weight per 24 hours.

26. The method according to any one of claims 15 to 21, wherein N-acetylcysteine (NAC), or a pharmaceutically acceptable salt or solvate thereof, is administered at 150 mg per kg of body weight per 24 hours.

27. The method according to any one of claims 15 to 26, wherein N-acetylcysteine (NAC), or a pharmaceutically acceptable salt or solvate thereof, is administered for 6 hours to 6 days or longer.

28. The method according to any one of claims 15 to 27, wherein N-acetylcysteine (NAC), or a pharmaceutically acceptable salt or solvate thereof, is administered for 2 days to 8 days.

29. The method according to any one of claims 15 to 28, wherein N-acetylcysteine (NAC), or a pharmaceutically acceptable salt or solvate thereof, is administered for 2 days to 6 days.

30. The method according to any one of claims 15 to 29, wherein N-acetylcysteine (NAC), or a pharmaceutically acceptable salt or solvate thereof, is administered for 2 days to 4 days.

31. The method according to any one of claims 1 to 9, wherein the pharmaceutically acceptable compound is an agent that can enhance the production of glutathione in vivo.

32. The method according to claim 31, wherein the agent is selected from one or more of lipoic acid, glycine, glutamate, or a derivative thereof, or a pharmaceutically acceptable salt or solvate thereof.

33. The method according to claim 31, wherein the agent is a substance that upregulates an enzyme involved in production of glutathione in vivo.

34. The method of claim 33, wherein the enzyme is selected from one or more of glutamate cysteine ligase, glutathione synthetase, and glutathione reductase.

35. The method according to any one of claims 1 to 34, wherein the pharmaceutically acceptable compound is administered in combination with an additional active agent.

36. The method according to claim 35, wherein the additional active agent comprises a therapeutic agent suitable for use against a coronavirus infection.

37. The method according to claim 36, wherein the therapeutic agent is selected from Nafamostat, Remdesivir, Aprotinin, Nelfinavir, or a pharmaceutically acceptable salt or solvate thereof.

38. The method according to claim 35, wherein the additional active agent comprises glycine or a derivative, pharmaceutically acceptable salt or solvate thereof.

39. The method according to claim 38, wherein the glycine or a derivative, pharmaceutically acceptable salt or solvate thereof is administered in an amount of 50 mg per day to 15 g per day.

40. The method according to claim 38 or 39, wherein the glycine or a derivative, pharmaceutically acceptable salt or solvate thereof is administered in an amount of 200 mg per day to 8 g per day.

41. The method according to any one of claims 38 to 40, wherein the glycine or a derivative, pharmaceutically acceptable salt or solvate thereof is administered in an amount of at least 500 mg per day.

42. The method according to claim 35, wherein the additional active agent comprises one or more of selenium, sodium selenite, selenium yeast, or a derivative, pharmaceutically acceptable salt or solvate thereof.

43. The method according to claim 42, wherein the one or more of selenium, sodium selenite, selenium yeast, or a derivative, pharmaceutically acceptable salt or solvate thereof is administered in an amount of elemental selenium of 50 μg per day to 400 μg per day.

44. The method according to claim 42 or claim 43, wherein the one or more of selenium, sodium selenite, selenium yeast, or a derivative, pharmaceutically acceptable salt or solvate thereof is administered in an amount of elemental selenium of 150 μg per day to 250 μg per day.

45. The method according to claim 35, wherein the additional active agent comprises one or more of zinc, zinc gluconate, or a derivative, pharmaceutically acceptable salt or solvate thereof.

46. The method according to claim 45, wherein the one or more of zinc, zinc gluconate, or a derivative, pharmaceutically acceptable salt or solvate thereof is administered in an amount of elemental zinc of 20 mg per day to 300 mg per day.

47. The method according to claim 45 or claim 46, wherein the zinc, zinc gluconate, or a derivative, pharmaceutically acceptable salt or solvate thereof is administered in an amount of elemental zinc of 100 mg per day to 200 mg per day.

48. The method according to any one of claims 35 to 47, wherein the additional active agent further comprises one or more of vitamin C, vitamin D, magnesium, and thiamine (vitamin B1), or a pharmaceutically acceptable salt or solvate thereof.

49. The method according to any one of claims 1 to 48, wherein the subject is elderly, having pneumonia, or is otherwise vulnerable.

50. The method according to claim 49, wherein the subject is elderly.

51. Use of an effective amount of a pharmaceutically acceptable compound in the manufacture of a medicament for preventing, treating and/or reducing the severity of a condition or disease associated with a coronavirus infection,
wherein the pharmaceutically acceptable compound is an antioxidant or a substance capable of increasing the level of glutathione in the subject.

52. An effective amount of a pharmaceutically acceptable compound for use in a method of preventing, treating and/or reducing the severity of a condition or disease associated with a coronavirus infection,
wherein the pharmaceutically acceptable compound is an antioxidant or a substance capable of increasing the level of glutathione in the subject.
